SEQUENCE LISTING (13 sequences)

SEQ ID NO: 1 - protein, 15 aa, synthetic construct
GGGGSGGGGSGGGGS

SEQ ID NO: 2 - protein, 112 aa, synthetic construct
ARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRT

SEQ ID NO: 3 - protein, 123 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAA

SEQ ID NO: 4 - protein, 126 aa, synthetic construct
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGIL

SEQ ID NO: 5 - protein, 126 aa, synthetic construct; identical to SEQ ID NO: 4

SEQ ID NO: 6 - protein, 20 aa, synthetic construct
ALPVTALLLPLALLLHAARP

SEQ ID NO: 7 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 8 - protein, 42 aa, synthetic construct
KRGRKKLLYIFKQPFMRPVQTTQEEDGCSCRFPEEEEGGCEL

SEQ ID NO: 9 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 776 aa, synthetic construct
ALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAAGGGGSGGGGSGGGGSARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGGGGSGGGGSGGGGSGGGGSGGGGSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 2328 bp, synthetic construct
  source 1..2328: mol_type = other DNA
gccctgcctgtgacagccctgctgctgcccctggccctgctgctgcacgccgcccgccccgaggtgcagctggtggaatctggcggcggactggtgcagcctggcggctccctgagactgtcctgtgctgctagtggctttaatatcaaagatacatacatccattgggtgaggcaggctccaggaaaaggcctggagtgggtggctagaatctatccaaccaatggctacactagatacgctgatagcgtgaagggcaggtttacaatctctgctgatactagcaaaaatactgcttatctgcagatgaatagcctgagggctgaggatacagcagtgtattattgtagcaggtggggaggcgatggattttatgctatggactattggggacagggaacactggtgacagtgagtagtgcagctgccggaggtggaggaagcggcggcggaggatctggcggagggggatctgctagacctgatatccagatgacacagagtccatcctcactgtctgcctcagttggagatagagtgactattacttgtagagcttcacaggatgtgaatacagctgtggcttggtatcagcagaaacctggaaaagctcctaagctgctgatctattctgctagctttctgtatagtggcgtgccttcaagattttcaggctctaggtcaggaactgattttacactgacaatctctagtctgcagcctgaggattttgctacatattattgtcagcagcattatacaacaccacctacatttggacagggaaccaaagtagagatcaaaagaacaggcggtggaggctctggaggcggaggcagtggaggaggaggatcaggcggaggaggaagcggaggcgggggaagtcaggtgcagttacagcagtctggccctggcctggtgacacctagtcagaccctgtctctgacctgtgctatttctggagatagtgtgagtagtaatagtgcaacatggaattggatcaggcagtctcctagtagaggcctggaatggctgggtagaacttattataggtctaaatggtataatgattatgctgtgtctgtgaagtctagaatgtctatcaatcctgatacctctaaaaatcagtttagcctgcagctgaatagtgtgactcctgaggatacagcagtgtactattgtgccagaggcatgatgacatattattacggaatggatgtgtggggccagggaacaacagtgaccgtgtcctctgggattctgggatctggaggaggcggcagcggcggaggaggtagtggaggcggaggatcacagcctgtgctgacccagagctcttctctgagcgccagtcctggagctagtgccagtctgacatgtacactgagatctggtatcaatgtgggcccttatagaatctattggtatcagcagaaacctggctcccctcctcagtacctgctgaattataaaagtgatagtgataaacagcagggatcaggcgtgccatctagattttctggaagcaaagatgctagcgctaatgccggtgtgctgttaatttctggcctgagatctgaagatgaggctgattattattgtatgatttggcatagttccgcagccgtgtttggaggcggcacacagctgacagtgctgaccacaacccctgcccccaggccccccacccccgcccctaccattgcctcccagcccctgtccctgcggcccgaggcctgccggcccgccgcaggaggcgccgtgcacacccgcggcctggacttcgcctgtgacatctacatttgggcccccctggccggcacatgcggcgtgctgctgctgagcctggtcatcaccctgtactgcaagaggggaagaaagaagctgctgtacattttcaagcagcccttcatgaggcccgtgcagacaacccaggaggaggatggctgtagctgcagattccccgaggaggaggagggcggctgcgagctgcgcgtgaagttttccaggtccgctgacgcccccgcctaccagcagggccagaaccagctgtacaacgagctgaacctgggcaggagggaggagtacgacgtgctggacaagagaaggggcagagaccccgagatgggcggcaagccccggaggaagaatccccaggagggcctgtacaacgagctgcagaaagataagatggctgaggcctattctgaaatcggcatgaagggagaaagaagaagaggaaaaggccacgatggcctgtatcagggactgagcactgccactaaagatacatatgatgccctgcacatgcaggccctgcctcccaga

SEQ ID NO: 12 - protein, 250 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAAGGGGSGGGGSGGGGSARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRT

SEQ ID NO: 13 - protein, 256 aa, synthetic construct
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVL

(19) United States
(12) Patent Application Publication
(10) Pub. No.: US 2024/0366671 A1
(43) Pub. Date: Nov. 7, 2024

(54) COMBINED HER2 AND MESO DUAL-TARGET CAR-T VECTOR, CONSTRUCTION METHOD THEREFOR AND APPLICATION THEREOF IN CANCER

(71) Applicant: SHANGHAI INSTITUTE OF BIOLOGICAL PRODUCTS CO., LTD., Shanghai (CN)

(72) Inventors: Fengting JIANG, Shanghai (CN); Jian LUO, Shanghai (CN); Mei ZHENG, Shanghai (CN); Yahong DING, Shanghai (CN); Feifei XIONG, Shanghai (CN); Xueying LIU, Shanghai (CN); Xu ZHOU, Shanghai (CN)

(21) Appl. No.: 18/683,176

(22) PCT Filed: Aug. 11, 2022
(86) PCT No.: PCT/CN2022/111866

§ 371 Date: Feb. 12, 2024

(30) Foreign Application Priority Data
Aug. 12, 2021 (CN) 202110925203.1

(51) Int. Cl. A61K 35/17; A61K 39/00; A61P 35/00; C07K 16/30; C07K 16/32; C12N 5/0783; C12N 15/86

(52) U.S. Cl. CPC A61K 35/17; A61K 39/4611; A61K 39/4631; A61K 39/464406; A61K 39/464468; A61P 35/00; C07K 16/30; C07K 16/32; C12N 5/0636; C12N 15/86; A61K 2239/13; A61K 2239/17; A61K 2239/21; C12N 2740/16043

(57) ABSTRACT

A combined HER2 and MESO dual-target CAR-T vector, a construction method therefor and an application thereof in cancer are provided; specifically, a bispecific chimeric antigen receptor (CAR) containing HER2 scFv and MESO scFv, a 4-1BB co-stimulatory domain, and a CD3 activation domain is provided. The bispecific CAR-T cell has a significant killing effect on HER2-positive target cells and MESO-positive target cells and can improve on the tumor-killing effect of T cells; the cytokines produced have a super-additive effect, and compared with a single-target CAR-T, can better clear tumor cells, alleviate antigen escape caused by tumor heterogeneity, and further strengthen the ability of CAR-T cells to kill tumors.

### TECHNICAL FIELD

The present invention relates to the field of biotechnology, and more specifically to combined HER2 and MESO dual-target CAR-T vectors and construction methods therefor and application in cancer.

### BACKGROUND

Cancer, which is an abnormal proliferation that occurs due to the loss of normal growth regulation of normal cells in the organism and can be accompanied by distant metastasis, has a high degree of malignancy and is a major killer threatening human life and health in today's society. Among them, pancreatic cancer is a kind of digestive tract tumor with high degree of malignancy, and it is one of the malignant tumors with the worst prognosis, and the 5-year survival rate is only 9%. Primary tumors of the pancreas mainly include three tissue sources: excrine, endocrine and mesenchymal tissues, among which exocrine pancreatic neoplasms account for more than 90% of pancreatic tumors, and 80% of them originate from the pancreas ductal epithelium. Due to its hidden physiological and anatomical location, it is not easy to be detected in the early stage and the best time for diagnosis and surgery is lost. The vast majority of patients are diagnosed with advanced or metastatic tumors, which has led to it becoming the fourth most common tumor that causes death from cancer. Pancreatic cancer is expected to become the second leading cause of cancer death in the United States by 2030. Clinical treatment is still based on traditional surgically removed combined with radiotherapy and chemotherapy. Due to the complexity and diversity of its causative factors, the best means of attacking the cancer has not been found. In recent years, tumor cell immunotherapy represented by chimeric antigen receptor-T cell (CAR-T) has brought new hope for the treatment of cancer.

CAR-T, as one of the emerging means of tumor immunotherapy, has become the focus and hotspot in the field of tumor therapy research in recent years. CAR-T is the use of genetic engineering technology to isolate the patient's own T-cells in vitro and express a single-chain antibody (ScFv) region that can specifically recognize the tumor surface antigen on its surface, so as to achieve the purpose of accurate targeting and killing of cancer cells. The gene-edited T cells express a CAR molecule, which mainly consists of three parts: the extracellular single-chain antibody region, the transmembrane region and the intracellular signaling region, which are involved in signal transmission and cascade amplification.

Currently, CAR-T therapy for solid tumors still presents many challenges, including: lack of ideal therapeutic targets, homing barriers, and persistent poor performance of CAR-T cells due to the immunosuppressive microenvironment.

Therefore, there is a need in this field to develop new CAR-T cells and therapeutic approaches for solid tumors, especially pancreatic cancer.

### SUMMARY OF THE INVENTION

It is an object of the present invention to provide a CAR-T cell and a therapeutic method for solid tumors, in particular pancreatic cancer.

A first object of the present invention:

To provide a tandem chimeric antigen receptor with a CAR structure comprising a HER2 single-chain antibody and a MESO single-chain antibody, referred to as HM CAR.

Wherein, the amino acid sequence of a CD8-derived signal peptide is shown in SEQ ID NO: 6;

Wherein, the HER2 single-chain antibody structure is a specific antigen-binding domain designed to target the pancreatic cancer surface tumor-associated antigen HER2, and the amino acid sequence of the HER2 single-chain antibody is shown in SEQ ID NO. 12.

Wherein the MESO single chain antibody structure is a specific antigen binding domain designed against the pancreatic cancer surface tumor-associated antigen MESO, and the amino acid sequence of the MESO single chain antibody is shown in SEQ ID NO.13.

The HER2 single-chain antibody and MESO single-chain antibody are linked by the hinge between single-chain antibodies Inner-Linker, and the amino acid sequence is shown in SEQ ID NO.1.

CD8α is a transmembrane region connecting the extracellular antigen-binding domain and the intracellular signaling domain that anchors the CAR structure to the T cell membrane, and its amino acid sequence is shown in SEQ ID NO.7.

4-1BB is a co-stimulatory domain that transduces proliferative signals and induces cytokine production, and its amino acid sequence is shown in SEQ ID NO.8.

CD3ζ is an intracellular signal transduction domain that transduces TCR-like signals to the intracellular when the extracellular region binds to the target antigen, activating T cells to exert targeted killing of tumor cells, and the amino acid sequence is shown in SEQ ID NO.9.

Further, the CAR structure is composed of Signal Peptide-HER2VL-(G4S)₃-HER2V_{H}-(G4S)₅-MESOV_{H}-(G4S)₃-MESOV_{L}-CD8α-4-1BB-CD3ζ.

A second object of the present invention:

To provide a dual-target CAR-T therapeutic vector against pancreatic cancer, consisting of two components, the lentiviral expression vector pLenti6.3/V5 and the HM CAR structure.

Among them, the pLenti6.3/V5 structure is schematically shown in FIG. 1: carrying the CAR gene as a vector plasmid, cPPT (Polypurine Tract) from the HIV-1 integrase gene, which increases the copy number of the lentivirus integrated into the host genome. The vector can be used to package, transduce and stably integrate lentivirus expression gene into the host's genome.

A third object of the present invention:

To provide a method for constructing a dual-target CAR-T therapeutic vector against pancreatic cancer.

The above CAR structure was synthesized by conventional biosynthesis method according to the gene sequence, and the synthesized CAR existed in PUC57 plasmid vector; the lentiviral expression vector pLenti6.3/V5 was purchased from Invitrogen, the PUC57 plasmid vector and the lentiviral expression vector were both double-enzymatically digested by BamHI and XhoI, and the digested products were separated by agarose gel electrophoresis separation, recovery of the target bands, the concentration of the vector and the target fragment is obtained, the two in accordance with the molar ratio of 1:5 for the connection and the transformation, plasmid extraction, and ultimately obtain the recombinant plasmid containing the structure of the chimeric antigen receptor, and ultimately obtain the recombinant plasmid containing the structure of the specific CAR.

A fourth object of the present invention:

To provide an application of a dual-target CAR-T therapeutic vector against pancreatic cancer, i.e., to provide a CAR-T cell.

Lentiviral packaging was carried out using a four-plasmid packaging system, three helper plasmids (pLP1, pLP2 plasmid, pLP/VSVG plasmid) were co-transfected with the lentiviral expression vector in HEK293 cells, and the viral fluid cultured for 48 h-55 h was collected. This viral fluid was concentrated and the viral titer was measured, and eventually infected T cells with MOI=15, and the CAR-T cells were finally obtained.

In a first aspect of the present invention, it provides a bispecific chimeric antigen receptor (CAR), having a structure as shown in Formula I below:

L-scFv1-I-scFv2-H-TM-C-CD3ζ (I)

wherein

each “-” is independently a linker peptide or peptide bond;

L is an optional signal peptide sequence;

I is a flexible linker;

H is an optional hinge region;

TM is a transmembrane domain;

C is co-stimulatory signaling molecule;

CD3ζ is a cytoplasmic signaling sequence derived from CD3ζ;

One of the two, scFv1 and scFv2, is an antigen-binding domain targeting HER2 and the other is an antigen-binding domain targeting MESO.

In another preferred example, the scFv1 is an antigen-binding domain targeting HER2 and the scFv2 is an antigen-binding domain targeting MESO.

In another preferred example, the antigen-binding domain targeting HER2 has a structure as shown in Formula A or Formula B below:

V_{H1}-V_{L1} (A);

V_{L1}-V_{H1} (B)

wherein V_{H1} is a heavy chain variable region; of an anti-HER2 antibody; V_{L1} is a light chain variable region of an anti-HER2 antibody; and “-” is a linker peptide or peptide bond.

In another preferred example, the structure of the antigen-binding domain targeting HER2 is shown in Formula B.

In another preferred example, the V_{H1} and V_{L1} are linked by a flexible linker (or linker peptide), the flexible linker (or linker peptide) is 1-4, preferably 2-4, more preferably 3-4 consecutive sequences as shown in GGGGS.

In another preferred example, the amino acid sequence of the heavy chain variable region of the anti-HER2 antibody is shown in SEQ ID NO:2 and the amino acid sequence of the light chain variable region of the anti-HER2 antibody is shown in SEQ ID NO:3.

In another preferred example, the structure of the antigen-binding domain targeting MESO is shown in Formula C or Formula D below:

V_{L2}-V_{H2} (C);

V_{H2}-V_{L2} (D)

wherein V_{L2} is a light chain variable region of an anti-MESO antibody; V_{H2} is a heavy chain variable region of an anti-MESO antibody; and “-” is a linker peptide or peptide bond.

In another preferred example, the structure of the antigen-binding domain targeting MESO is shown in Formula D.

In another preferred example, the V_{L2} and V_{H2} are linked by a flexible linker (or linker peptide), the flexible linker (or linker peptide) is 1-4, preferably 2-4, more preferably 3-4 consecutive sequences as shown in GGGGS.

In another preferred example, the amino acid sequence of the heavy chain variable region of the anti-MESO antibody is shown in SEQ ID NO:4 and the amino acid sequence of the light chain variable region of the anti-MESO antibody is shown in SEQ ID NO:5.

In another preferred example, the scFv1 and/or scFv2 are murine, human, human and murine chimeric, or fully humanized single chain antibody variable region fragments.

In another preferred example, the sequence of flexible linker I comprises 2-6, preferably 2-4, more preferably 3-4 consecutive sequences as shown in GGGGS.

In another preferred example, the L is a signal peptide of a protein selected from the group consisting of: CD8, CD28, GM-CSF, CD4, CD137, and a combination thereof.

In another preferred example, the L is a signal peptide of CD8 origin.

In another preferred example, the amino acid sequence of L is as shown in SEQ ID NO: 6.

In another preferred example, the TM is a transmembrane region of a protein selected from the group consisting of CD8α, CD28, CD3 epsilon, CD45, CD4, CD5, CD8, CD9, CD16, GD2, CD33, CD37, CD64, CD80, CD86, CD134, CD137, CD154, and a combination thereof.

In another preferred example, the TM is a transmembrane region of CD8α origin.

In another preferred example, the sequence of the TM is as shown in SEQ ID NO: 7.

In another preferred example, the C is a co-stimulatory signaling molecule of a protein selected from the group consisting of: 4-1BB (CD137), OX40, CD2, CD7, CD27, CD28, CD30, CD40, CD70, CD134, PD1, Dap10, CDS, ICAM-1, LFA-1 (CD11a/CD18), ICOS (CD278), NKG2D, GITR, TLR2, and a combination thereof.

In another preferred example, the C is a co-stimulatory signaling molecule of 4-1BB origin.

In another preferred example, the co-stimulatory signaling molecule of 4-1BB origin has the amino acid sequence as shown in SEQ ID NO: 8.

In another preferred example, the amino acid sequence of CD32 is shown in SEQ ID NO: 9.

In another preferred example, the amino acid sequence of the chimeric antigen receptor is shown in SEQ ID NO: 10.

In a second aspect of the present invention, it provides a nucleic acid molecule encoding the chimeric antigen receptor (CAR) as described in the first aspect of the present invention.

In another preferred example, the nucleic acid molecule is isolated.

In another preferred example, the nucleic acid molecule has a nucleotide sequence as shown in SEQ ID NO:11.

In a third aspect of the present invention, it provides a vector comprising a nucleic acid molecule as described in the second aspect of the present invention.

In another preferred example, the vector is selected from the group consisting of: DNA, RNA, plasmid, lentiviral vector, adenoviral vector, adeno-associated viral vector (AAV), retroviral vector, transposon, and a combination thereof.

In another preferred example, the vector is selected from the group consisting of: plasmid, viral vector.

In another preferred example, the vector is in the form of a viral particle.

In another preferred example, the vector is a lentiviral vector.

In a fourth aspect of the present invention, it provides a host cell containing a vector as described in the third aspect of the present invention or a chromosome incorporating an exogenous nucleic acid molecule as described in the second aspect of the present invention or expressing a CAR as described in the first aspect of the present invention.

In another preferred example, the cell is an isolated cell.

In another preferred example, the cell is a genetically engineered cell.

In another preferred example, the cell is a mammalian cell.

In another preferred example, the cell is from a human or non-human mammal (e.g. mouse).

In another preferred example, the cell comprises T cell, NK cell.

In another preferred example, the cell is CAR-T cell or CAR-NK cell, preferably CAR-T cell.

In another preferred example, the cell targets both HER2 and MESO.

In a fifth aspect of the present invention, it provides a formulation comprising a chimeric antigen receptor as described in the first aspect of the present invention, a nucleic acid molecule as described in the second aspect of the present invention, a vector as described in the third aspect of the present invention, or a host cell as described in the fourth aspect of the present invention, as well as a pharmaceutically acceptable carrier, diluent or excipient.

In another preferred example, the formulation is a liquid formulation.

In another preferred example, the dosage form of the formulation is an injection.

In another preferred example, the concentration of the cell in the formulation is 1×10⁵-1×10⁸ cells/ml, preferably 1×10⁷-1×10⁸ cells/ml.

In a sixth aspect of the present invention, it provides a use of the chimeric antigen receptor as described in the first aspect of the present invention, the nucleic acid molecule as described in the second aspect of the present invention, the vector as described in the third aspect of the present invention, or the host cell as described in the fourth aspect of the present invention, or the formulation as described in the fifth aspect of the present invention for the preparation of a medicament or a formulation for the prevention and/or treatment of a cancer or a tumor.

In another preferred example, the tumor comprises a solid tumor.

In another preferred example, the solid tumor is selected from the group consisting of: pancreatic cancer, gastric cancer, peritoneal metastasis in gastric cancer, liver cancer, renal tumor, lung cancer, carcinoma of small intestine, bone cancer, prostate cancer, colorectal cancer, breast cancer, carcinoma of large intestine, cervical cancer, ovarian cancer, adrenal tumor, bladder tumors, non-small cell lung cancer (NSCLC), brain glioma, endometrial cancers, and a combination thereof.

In another preferred example, the tumor comprises a HER2 and/or MESO positive solid tumor.

In a seventh aspect of the present invention, it provides a kit for preparing a host cell as described in the fourth aspect of the present invention comprising a container, and the nucleic acid molecule as described in the second aspect of the present invention, or the vector as described in the third aspect of the present invention in the container.

In an eighth aspect of the present invention, it provides a method of preparing an engineered immune cell, the immune cell expresses the CAR as described in the first aspect of the present invention, the method comprises the steps of:

(a) providing an immune cell to be engineered; and

(b) transducing the nucleic acid molecule as described in the second aspect of the present invention or the vector as described in the third aspect of the present invention into the immune cell, thereby obtaining the engineered immune cell.

In another preferred example, the engineered immune cell is a CAR-T cell or a CAR-NK cell.

In another preferred example, the method further comprises the step of detecting the function and efficacy of engineered immune cell obtained.

In another preferred example, the immune cell comprises T cell, NK cell, macrophage.

In a ninth aspect of the present invention, it provides a use of the host cell described in the fourth aspect of the present invention, or the formulation as described in the fifth aspect of the present invention for the prevention and/or treatment of a cancer or a tumor.

In a tenth aspect of the present invention, it provides a method of treating a disease comprising administering to a subject in need of treatment an appropriate amount of the vector as described in the third aspect of the present invention, the host cell as described in the fourth aspect of the present invention, or the formulation as described in the fifth aspect of the present invention.

In another preferred example, the disease is a cancer or a tumor.

It should be understood that within the scope of the present invention, the various technical features of the present invention above and the various technical features specifically described hereinafter (as in the embodiments) may be combined with each other to constitute a new or preferred technical solution. Due to space limitations, it is not repeated here.

### DESCRIPTION OF THE FIGURES

FIG. 1 shows a schematic diagram of the structure of the pLenti6.3/V5 vector plasmid;

FIG. 2 shows the flow chart of the present invention;

FIG. 3 shows the agarose gel electrophoresis results of the HM CAR gene constructed in the pLenti6.3/V5 vector after identification by BamHI and Xhol double digestion;

FIG. 4 shows the graph of flow cytometric analysis detection results of HER2 and MESO antigen expression in pancreatic cancer cells SW-1990 (4-1) and ASPC-1 (4-2);

FIG. 5 shows the results of transduction efficiency detection of HM CAR-T cells;

FIG. 6 shows a graph of the results of RTCA monitoring the killing of pancreatic cancer cells by HM CAR-T cells;

FIG. 7 shows a schematic diagram of the CAR structure;

FIG. 8 shows that the therapeutic effect of HM CAR-T cells is superior to that of single-target CAR-T cells.

### DETAILED DESCRIPTION

The present inventors, after extensive and in-depth research, have for the first time accidentally discovered a bispecific CAR targeting HER2 and MESO, the bispecific CAR comprises HER2 scFv and MESO scFv, as well as 4-1BB co-stimulatory domains and CD3 activation domains. Experiments have shown that the bispecific CAR-T cells of the present invention improve the tumor killing effect of T cells, the cytokines produced have a super-additive effect, and can better clear tumor cells and attenuating antigenic escape caused by tumor heterogeneity compared to single-targeted CAR-T, which further strengthens the ability of the CAR-T cells to kill tumors. On this basis, the present inventor has completed the present invention.

### Terms

In order that the present disclosure may be more readily understood, certain terms are first defined. As used in this application, each of the following terms shall have the meaning given below unless otherwise expressly provided herein. Other definitions are set forth throughout the application.

The term “about” may refer to a value or composition that is within an acceptable margin of error for a particular value or composition as determined by one of ordinary skill in the art, which will depend in part on how the value or composition is measured or determined.

The term “administration” refers to the physical introduction of the product of the invention into a subject using any of a variety of methods and delivery systems known to those of ordinary skill in the art, including intravenously, intramuscularly, subcutaneously, intraperitoneally, spinal cord, or other parenteral routes of administration, e.g., by injection or infusion.

The term “antibody” (Ab) shall include, but is not limited to, an immunoglobulin which specifically binds an antigen and comprises at least two heavy (H) chains and two light (L) chains interconnected by disulfide bonds, or antigen-binding portions thereof. Each H chain comprises a heavy chain variable region (abbreviated herein as VH) and a heavy chain constant region. The heavy chain constant region contains three constant domains CH1, CH2 and CH3. Each light chain contains a light chain variable region (abbreviated herein as VL) and a light chain constant region. The light chain constant region contains a constant domain CL. The VH and VL regions can be further subdivided into highly variable regions known as complementary decision regions (CDRs), which are scattered with more conserved regions known as framework regions (FRs). Each VH and VL contains three CDRs and four FRs arranged from amino terminus to carboxy terminus in the following order: FR1, CDR1, FR2, CDR2, FR3, CDR3, FR4. The heavy and light chain variable regions contain binding domains that interact with antigens.

It should be understood that the amino acid names herein are identified using the internationally recognized single English letter identifiers, and the three-letter abbreviations of the amino acid names corresponding thereto are Ala (A), Arg (R), Asn (N), Asp (D), Cys (C), Gln (Q), Glu (E), Gly (G), His (H), Ile (I), Leu (L), Lys (K), Met (M), Phe (F), Pro (P), Ser(S), Thr (T), Trp (W), Tyr (Y), Val (V).

### HER2 and MESO Antigens

HER2, also known as ErbB-2/neu, is located on the long arm of chromosome 17 (17q12-21.32) and can encode a 185 kDa transmembrane receptor protein with tyrosine kinase activity. Under physiological conditions, the binding of the receptor to the ligand induces HER2 dimerization and activates downstream signaling pathways such as MAPK and PI3K, promoting cell proliferation and preventing apoptosis. When HER2 is overexpressed on the cell surface, cells undergo overproliferation and malignant metastasis. Therefore, by blocking the activated HER2 signaling pathway, tumor cell proliferation is inhibited, apoptosis pathway is initiated, and programmed tumor cell death is promoted. About 20% of primary invasive ductal carcinomas of the breast have HER2 overexpression; the positive rate of HER2 expression in patients in the androgen-independent prostate cancer (AIPC) group is higher than that in the benign prostatic hyperplasia group and the androgen-dependent prostate cancer group; and positive expression is also found in about 20% to 60% of patients with pancreatic ductal adenocarcinoma (PDAC). Therefore, HER2 could be an ideal target to treat tumors with high positive expression rates.

Mesothelin (MESO) is a glycoprotein anchored by glycosylphosphatidylinositol (GPI) with a molecular weight of about 40 KD. it is not expressed in normal tissue cells or is expressed in trace amounts in mesothelial cells. Almost 100% of pancreatic cancers are positive for MSLN, and other tumors such as extrahepatic cholangiocarcinoma (95%), endometrial carcinoma (89%), triple-negative breast cancer (66%), esophageal carcinoma (46%), colorectal carcinoma (30%), and cervical carcinoma (25%) are expressed to varying degrees. Abnormal MSLN expression leads to further tumor deterioration, which occurs through two main pathways: first, activation of intracellular signaling pathway through GPI and continuous activation of NF-κB, MAPK, and PI3-kinase signaling pathway, which promotes the proliferation of tumor cells and strengthens their anti-apoptotic ability; second, high affinity binding of MSLN to the receptor CA125/MUC16, which promotes the heterogeneous adhesion between cells, leading to the spread and metastasis of tumor cells. Therefore, according to the relatively high expression of MSLN, it can be selected as a more specific target for CAR-T cell therapy.

### Bispecific Chimeric Antigen Receptor Targeting HER2 and MESO

Cellular immunotherapy is an emerging tumor treatment modality with remarkable efficacy and is a novel therapeutic approach for autoimmune anti-cancer. It is a method of using biotechnology and biologics to stimulate and enhance the body's autoimmune function by using immune cells collected from the patient's body to be cultured and expanded in vitro and then infused back into the patient's body, so as to achieve the purpose of treating tumors. Those skilled in the art have been committed to developing new cellular immunotherapy to improve the effectiveness of cellular immunotherapy and to reduce their side effects.

The present invention presents a rational and optimized single-chain design and system, i.e., a combined bispecific CAR, wherein said CAR can be efficiently integrated into primary human T-cells and can target both HER2 and MESO when the T-cells are activated. The CAR-T cells of the present invention are capable of recognizing two antigens (HER2 and MESO), which is a very effective and potential method for preventing antigen escape.

The use of bi-directionally-targeted HER2 and MESO CAR of the present invention can better clear tumor cells and alleviate the antigen escape phenomenon caused by tumor heterogeneity compared to the CAR targeting a single antigen, and further enhance the ability of CAR-T cells to kill tumors with cytokine synergy effect. In addition, since HER2 and MESO are not expressed at uniform levels in tumor cells, dual-targeted CAR-T has a wider therapeutic range. CAR-T that simultaneously targets HER2 and MESO on the surface of tumor cells reduces the possibility of antigen escape due to down-regulation or deletion of a single surface antigen.

Bispecificity refers to the fact that the same CAR can specifically bind and immunorecognize two different antigens, and the CAR can generate an immune response by binding either antigen.

The HER2 and MESO bispecific CAR of the present invention is a single structure comprising anti-HER2 and anti-MESO scFv. Wherein the CAR comprises HER2 scFv and MESO scFv, and the amino acid sequences, sequencing, and hinges of the HER2 scFv and the MESO scFv are the primary influences on its function.

Specifically, the chimeric antigen receptor (CAR) of the present invention includes an extracellular domain, a transmembrane domain, and an intracellular domain. The extracellular domain includes a target-specific binding element (also known as an antigen-binding domain). The intracellular domains include the co-stimulatory signaling region and the ζ-chain portion. The co-stimulatory signaling region refers to a portion of the intracellular domain that includes co-stimulatory molecules. Co-stimulatory molecules are cell surface molecules that are required for an effective response of lymphocytes to antigens, rather than antigen receptors or their ligands.

A linker may be incorporated between the extracellular domain and the transmembrane domain of the CAR, or between the cytoplasmic domain and the transmembrane domain of the CAR. As used herein, the term “linker” generally refers to any oligopeptide or polypeptide that serves to connect the transmembrane domain to the extracellular or cytoplasmic domain of a polypeptide chain. The linker may comprise from 0 to 300 amino acids, preferably from 2 to 100 amino acids and most preferably from 3 to 50 amino acids.

In a preferred embodiment of the present invention, the extracellular domain of the CAR provided herein comprises an antigen-binding domain targeting the combination of HER2 and MESO. The CAR of the present invention when expressed in T cells is capable of antigen recognition based on antigen binding specificity. When it binds its associated antigen, it affects tumor cells, resulting in the tumor cells not growing, being prompted to die, or otherwise being affected, and resulting in a reduction or elimination of the patient's tumor load. The antigen-binding domain is preferably fused to an intracellular domain from one or more of the co-stimulatory molecule and the ζ-chain. Preferably, the antigen-binding domain is fused to an intracellular domain of a combination of a 4-1BB signaling domain, and a CD3ζ signaling domain.

As used herein, the term “antigen-binding domain”, “single chain antibody fragment” refers to a Fab fragment, a Fab′ fragment, an F(ab′)₂ fragment, or a single Fv fragment having antigen-binding activity. Fv antibody contains the smallest antibody fragment that contains a heavy chain variable region of the antibody, a light chain variable region, but no constant region, and has all of the antigen binding sites. Generally, the Fv antibody also contains a polypeptide junction between the VH and VL domains and is capable of forming the structure required for antigen binding. The antigen-binding domain is typically a scFv (single-chain variable fragment). scFv is typically ⅙ the size of a complete antibody. a single-chain antibody is preferably a sequence of a single amino acid chain encoded by a single nucleotide chain. As a preferred embodiment of the present invention, said scFv comprises an antibody that specifically recognizes HER2 and MESO.

For the hinge region and the transmembrane region (transmembrane domain), the CAR may be designed to comprise a transmembrane domain fused to the extracellular domain of the CAR. In one embodiment, a transmembrane domain that is naturally associated with one of the domains in the CAR is used. In some examples, the transmembrane domain may be selected, or modified by amino acid substitutions, to avoid binding such a domain to the transmembrane domain of the same or a different surface membrane protein, thereby minimizing interaction with other members of the receptor complex.

The intracellular domains in the CAR of the present invention include the signaling domain of 4-1BB and the signaling domain of CD3ζ.

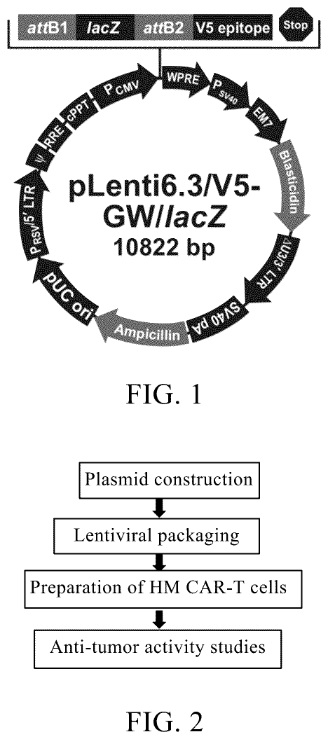

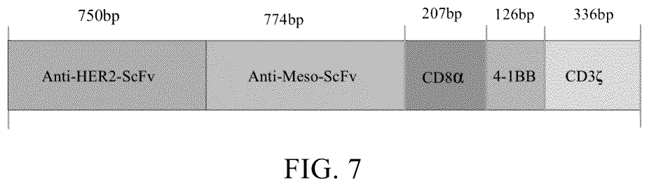

Preferably, the structure of the CAR of the present invention comprises, in order, a signaling peptide sequence (also known as a lead sequence), an antigen recognition sequence (antigen-binding domain), optionally a hinge region, a transmembrane region, a co-stimulatory factor signaling region, and a CD3zeta signaling region (ζ-chain portion), The CAR gene-containing vector plasmid is shown in FIG. 1 as a vector plasmid carrying the CAR gene, from the HIV-1 integrase gene of cPPT (Polypurine Tract), which increases the copy number of lentivirus integrated into the host genome, the CAR structure of the present invention is shown in FIG. 7.

In another preferred example, the CAR of the present invention is a HM CAR. wherein the antigen-binding domain targeting HER2 comprises a single-chain variable region heavy chain sequence (SEQ ID NO: 2) and a single-chain variable region light chain (VL) sequence (SEQ ID NO: 3) of HER2 antibody origin.

Amino acid sequence of a single chain variable region heavy chain (VH) of HER2 antibody origin:

(SEQ ID NO: 2)
ARPDIQMTQSPSSLSASVGDRVTITCRASQDVNTAVAWYQQKPGK
APKLLIYSASFLYSGVPSRFSGSRSGTDFTLTISSLQPEDFATYY
CQQHYTTPPTFGQGTKVEIKRT

HER2 antibody-derived single-chain variable region light chain (VL) sequence:

(SEQ ID NO: 3)
EVQLVESGGGLVQPGGSLRLSCAASGFNIKDTYIHWVRQAPGKGL
EWVARIYPTNGYTRYADSVKGRFTISADTSKNTAYLQMNSLRAED
TAVYYCSRWGGDGFYAMDYWGQGTLVTVSSAAA

HER2 single chain antibody HER2-ScFv sequence (HER2V_{L}-(G4S)₃-HER2V_{H})

(SEQ ID NO: 12)
EVQLVESGGGLVQPGGSLRLSCAASGFNIKDTYIHWVRQAPGKGL
EWVARIYPTNGYTRYADSVKGRFTISADTSKNTAYLQMNSLRAED
TAVYYCSRWGGDGFYAMDYWGQGTLVTVSSAAAGGGGSGGGGSGG
GGSARPDIQMTQSPSSLSASVGDRVTITCRASQDVNTAVAWYQQK
PGKAPKLLIYSASFLYSGVPSRFSGSRSGTDFTLTISSLQPEDFA
TYYCQQHYTTPPTFGQGTKVEIKRT

The antigen-binding domain targeting MESO comprises a MESO antibody-derived single-chain variable region heavy chain sequence (SEQ ID NO: 4) and a single-chain variable region light chain sequence (SEQ ID NO: 5).

MESO antibody-derived single-chain variable region heavy chain (VH) sequence:

(SEQ ID NO: 4)
QVQLQQSGPGLVTPSQTLSLTCAISGDSVSSNSATWNWIRQSPSR
GLEWLGRTYYRSKWYNDYAVSVKSRMSINPDTSKNQFSLQLNSVT
PEDTAVYYCARGMMTYYYGMDVWGQGTTVTVSSGIL

MESO antibody-derived single-chain variable region light chain (VL) sequence:

(SEQ ID NO: 5)
QVQLQQSGPGLVTPSQTLSLTCAISGDSVSSNSATWNWIRQSPSR
GLEWLGRTYYRSKWYNDYAVSVKSRMSINPDTSKNQFSLQLNSVT
PEDTAVYYCARGMMTYYYGMDVWGQGTTVTVSSGIL

MESO single chain antibody MESO-ScFv sequence (MESOV_{H}-(G4S)₃-MESOV_{L})

(SEQ ID NO: 13)
QVQLQQSGPGLVTPSQTLSLTCAISGDSVSSNSATWNWIRQSPSR
GLEWLGRTYYRSKWYNDYAVSVKSRMSINPDTSKNQFSLQLNSVT
PEDTAVYYCARGMMTYYYGMDVWGQGTTVTVSSGILGGGGSGGGG
SGGGGSQPVLTQSSSLSASPGASASLTCTLRSGINVGPYRIYWYQ
QKPGSPPQYLLNYKSDSDKQQGSGVPSRFSGSKDASANAGVLLIS
GLRSEDEADYYCMIWHSSAAVFGGGTQLTVL

Specifically, the sequences of the other components in the CAR of the present invention are as follows:

The signaling peptide is of CD8 origin:

(SEQ ID NO: 6)
ALPVTALLLPLALLLHAARP

The sequence of the linker between the heavy and light chains of the single-stranded variable region (i.e., flexible linker I) is:

Amino acid sequence:
(SEQ ID NO: 1)
GGGGSGGGGSGGGGS

The transmembrane region is a CD8α-derived transmembrane region sequence:

(SEQ ID NO.: 7)
TTTPAPRPPTPAPTIASQPLSLRPEACRPAAGGAVHTRGLDFACD
IYIWAPLAGTCGVLLLSLVITLYC

The co-stimulatory factor signaling region is derived from the sequence of the intracellular signaling motif of 4-1BB:

(SEQ ID NO.: 8)
KRGRKKLLYIFKQPFMRPVQTTQEEDGCSCRFPEEEEGGCEL

Sequence of the signaling domain of CD3ζ:

(SEQ ID NO: 9)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR

In a preferred embodiment, the complete nucleic acid sequence and amino acid sequence of the CAR constructed by the present invention is shown below:

Complete nucleic acid sequence of the HM CAR
(SEQ ID NO. 11)
GCCCTGCCTGTGACAGCCCTGCTGCTGCCCCTGGCCCTGCTGCTG
CACGCCGCCCGCCCCGAGGTGCAGCTGGTGGAATCTGGCGGCGGA
CTGGTGCAGCCTGGCGGCTCCCTGAGACTGTCCTGTGCTGCTAGT
GGCTTTAATATCAAAGATACATACATCCATTGGGTGAGGCAGGCT
CCAGGAAAAGGCCTGGAGTGGGTGGCTAGAATCTATCCAACCAAT
GGCTACACTAGATACGCTGATAGCGTGAAGGGCAGGTTTACAATC
TCTGCTGATACTAGCAAAAATACTGCTTATCTGCAGATGAATAGC
CTGAGGGCTGAGGATACAGCAGTGTATTATTGTAGCAGGTGGGGA
GGCGATGGATTTTATGCTATGGACTATTGGGGACAGGGAACACTG
GTGACAGTGAGTAGTGCAGCTGCCGGAGGTGGAGGAAGCGGCGGC
GGAGGATCTGGCGGAGGGGGATCTGCTAGACCTGATATCCAGATG
ACACAGAGTCCATCCTCACTGTCTGCCTCAGTTGGAGATAGAGTG
ACTATTACTTGTAGAGCTTCACAGGATGTGAATACAGCTGTGGCT
TGGTATCAGCAGAAACCTGGAAAAGCTCCTAAGCTGCTGATCTAT
TCTGCTAGCTTTCTGTATAGTGGCGTGCCTTCAAGATTTTCAGGC
TCTAGGTCAGGAACTGATTTTACACTGACAATCTCTAGTCTGCAG
CCTGAGGATTTTGCTACATATTATTGTCAGCAGCATTATACAACA
CCACCTACATTTGGACAGGGAACCAAAGTAGAGATCAAAAGAACA
GGCGGTGGAGGCTCTGGAGGCGGAGGCAGTGGAGGAGGAGGATCA
GGCGGAGGAGGAAGCGGAGGCGGGGGAAGTCAGGTGCAGTTACAG
CAGTCTGGCCCTGGCCTGGTGACACCTAGTCAGACCCTGTCTCTG
ACCTGTGCTATTTCTGGAGATAGTGTGAGTAGTAATAGTGCAACA
TGGAATTGGATCAGGCAGTCTCCTAGTAGAGGCCTGGAATGGCTG
GGTAGAACTTATTATAGGTCTAAATGGTATAATGATTATGCTGTG
TCTGTGAAGTCTAGAATGTCTATCAATCCTGATACCTCTAAAAAT
CAGTTTAGCCTGCAGCTGAATAGTGTGACTCCTGAGGATACAGCA
GTGTACTATTGTGCCAGAGGCATGATGACATATTATTACGGAATG
GATGTGTGGGGCCAGGGAACAACAGTGACCGTGTCCTCTGGGATT
CTGGGATCTGGAGGAGGCGGCAGCGGCGGAGGAGGTAGTGGAGGC
GGAGGATCACAGCCTGTGCTGACCCAGAGCTCTTCTCTGAGCGCC
AGTCCTGGAGCTAGTGCCAGTCTGACATGTACACTGAGATCTGGT
ATCAATGTGGGCCCTTATAGAATCTATTGGTATCAGCAGAAACCT
GGCTCCCCTCCTCAGTACCTGCTGAATTATAAAAGTGATAGTGAT
AAACAGCAGGGATCAGGCGTGCCATCTAGATTTTCTGGAAGCAAA
GATGCTAGCGCTAATGCCGGTGTGCTGTTAATTTCTGGCCTGAGA
TCTGAAGATGAGGCTGATTATTATTGTATGATTTGGCATAGTTCC
GCAGCCGTGTTTGGAGGCGGCACACAGCTGACAGTGCTGACCACA
ACCCCTGCCCCCAGGCCCCCCACCCCCGCCCCTACCATTGCCTCC
CAGCCCCTGTCCCTGCGGCCCGAGGCCTGCCGGCCCGCCGCAGGA
GGCGCCGTGCACACCCGCGGCCTGGACTTCGCCTGTGACATCTAC
ATTTGGGCCCCCCTGGCCGGCACATGCGGCGTGCTGCTGCTGAGC
CTGGTCATCACCCTGTACTGCAAGAGGGGAAGAAAGAAGCTGCTG
TACATTTTCAAGCAGCCCTTCATGAGGCCCGTGCAGACAACCCAG
GAGGAGGATGGCTGTAGCTGCAGATTCCCCGAGGAGGAGGAGGGC
GGCTGCGAGCTGCGCGTGAAGTTTTCCAGGTCCGCTGACGCCCCC
GCCTACCAGCAGGGCCAGAACCAGCTGTACAACGAGCTGAACCTG
GGCAGGAGGGAGGAGTACGACGTGCTGGACAAGAGAAGGGGCAGA
GACCCCGAGATGGGCGGCAAGCCCCGGAGGAAGAATCCCCAGGAG
GGCCTGTACAACGAGCTGCAGAAAGATAAGATGGCTGAGGCCTAT
TCTGAAATCGGCATGAAGGGAGAAAGAAGAAGAGGAAAAGGCCAC
GATGGCCTGTATCAGGGACTGAGCACTGCCACTAAAGATACATAT
GATGCCCTGCACATGCAGGCCCTGCCTCCCAGA
Complete amino acid sequence of HM CAR
(SEQ ID NO. 10)
ALPVTALLLPLALLLHAARPEVOLVESGGGLVQPGGSLRLSCAAS
GFNIKDTYIHWVRQAPGKGLEWVARIYPTNGYTRYADSVKGRFTI
SADTSKNTAYLQMNSLRAEDTAVYYCSRWGGDGFYAMDYWGQGTL
VTVSSAAAGGGGSGGGGSGGGGSARPDIQMTQSPSSLSASVGDRV
TITCRASQDVNTAVAWYQQKPGKAPKLLIYSASFLYSGVPSRFSG
SRSGTDFTLTISSLQPEDFATYYCQQHYTTPPTFGQGTKVEIKRT
GGGGGGGGSGGGGSGGGGSGGGGSQVQLQQSGPGLVTPSQTLSLT
CAISGDSVSSNSATWNWIRQSPSRGLEWLGRTYYRSKWYNDYAVS
VKSRMSINPDTSKNQFSLQLNSVTPEDTAVYYCARGMMTYYYGMD
VWGQGTTVTVSSGILGSGGGGSGGGGGGGGSQPVLTQSSSLSASP
GASASLTCTLRSGINVGPYRIYWYQQKPGSPPQYLLNYKSDSDKQ
QGSGVPSRFSGSKDASANAGVLLISGLRSEDEADYYCMIWHSSAA
VFGGGTQLTVLTTTPAPRPPTPAPTIASQPLSLRPEACRPAAGGA
VHTRGLDFACDIYIWAPLAGTCGVLLLSLVITLYCKRGRKKLLYI
FKQPFMRPVQTTQEEDGCSCRFPEEEEGGCELRVKFSRSADAPAY
QQGQNQLYNELNLGRREEYDVLDKRRGRDPEMGGKPRRKNPQEGL
YNELQKDKMAEAYSEIGMKGERRRGKGHDGLYQGLSTATKDTYDA
LHMQALPPR

### Chimeric Antigen Receptor T Cells (CAR-T Cells)

As used herein, the terms “CAR-T cell”, “CAR-T”, “CART”, “CAR-T cell of the present invention” all refer to CAR-T cells that target both HER2 and MESO as described in the fourth aspect of the present invention. Specifically the CAR structure of said CAR-T cells sequentially comprises an anti-HER2 scFv, an anti-MESO scFv, a transmembrane region, and an intracellular T-cell signaling region, and the anti-HER2 scFv and the MESO scFv are connected by a plurality of repeating structure (G4S) peptides. Compared with CAR-Ts targeting a single antigen, CAR-T cells recognizing both targets can better clear tumor cells and attenuate the antigen escape phenomenon caused by tumor heterogeneity, further enhancing the ability of CAR-T cells to kill tumors.

### Vector

The nucleic acid sequence encoding the desired molecule may be obtained using recombinant methods known in the art, such as, for example, by screening libraries from cells expressing the gene, by obtaining the gene from vectors known to include the gene, or by direct isolation of the gene from cells and tissues comprising the gene using standard techniques. Optionally, the gene of interest may be produced synthetically.

The present invention also provides vectors in which the expression cassettes of the present invention are inserted. Vectors derived from retroviruses, such as lentiviruses, are suitable tools for realizing long-term gene transfer, as they allow long-term, stable integration of the transgene and its proliferation in daughter cells. Lentiviral vectors have advantages over vectors derived from oncogenic retroviruses such as murine leukemia virus because they can transduce non-proliferating cells such as hepatocytes. They also have the advantage of low immunogenicity.

In brief summary, it is generally operable to ligate an expression cassette or nucleic acid sequence of the invention to a promoter and incorporate it into an expression vector. The vectors are suitable for replication and integration into eukaryotic cells. Typical cloning vectors contain transcriptional and translational terminators, initial sequences and promoters that can be used to regulate the expression of desired nucleic acid sequences.

The expression constructs of the present invention may also be utilized in standard gene delivery schemes for nucleic acid immunization and gene therapy. Methods of gene delivery are known in the art. See, e.g., U.S. Pat. Nos. 5,399,346, 5,580,859, 5,589,466, which are hereby incorporated by reference in their entirety. In another embodiment, the present invention provides gene therapy vectors.

The nucleic acid may be cloned into many types of vectors. For example, the nucleic acid may be cloned into such vectors, which include, but are not limited to, plasmids, phages, phage derivatives, animal viruses, and cosmid. Specific vectors of interest include expression vectors, replication vectors, probe generation vectors, and sequencing vectors.

Further, the expression vectors may be provided to the cells in the form of viral vectors. Viral vector technology is well known in the art and described in, for example, Sambrook et al. (2001, Molecular Cloning: A Laboratory Manual, Cold Spring Harbor Laboratory, New York) and other virology and molecular biology manuals. Viruses that may be used as vectors include, but are not limited to, retroviruses, adenoviruses, adeno-associated virus, herpesviruses, and lentiviruses. Typically, suitable vectors comprise a replication start, a promoter sequence, a convenient restriction enzyme site, and one or more selectable markers that functions in at least one organism (e.g., WO01/96584; WO01/29058; and U.S. Pat. No. 6,326,193).

Many virus-based systems have been developed for gene transfer into mammalian cells. For example, retroviruses provide a convenient platform for use in gene delivery systems. Selected genes can be inserted into a vector and packaged into retroviral particles using techniques known in the art. The recombinant virus may then be isolated and delivered to the subject cell in vivo or ex vitro. Many retroviral systems are known in the art. In some embodiments, an adenoviral vector is used. Many adenoviral vectors are known in the art. In one embodiment, a lentiviral vector is used.

Additional promoter elements, such as enhancers, can regulate the frequency of transcription initiation. Typically, these are located in a 30-110 bp region upstream of the start site, although it has recently been shown that many promoters also contain functional elements downstream of the start site. The spacing between promoter elements is often flexible to maintain promoter function when the element is inverted or moved relative to another. In the thymidine kinase (tk) promoter, the spacing between promoter elements can be increased by 50 bp apart before activity begins to decline. Depending on the promoter, exhibit individual elements can act cooperatively or independently to initiate transcription.

An example of a suitable promoter is the immediate early cytomegalovirus (CMV) promoter sequence. This promoter sequence is a strong constitutive promoter sequence capable of driving high level expression of any polynucleotide sequence operably linked thereto. Another example of a suitable promoter is elongation growth factor-1α (EF-1α). However, other constitutive promoter sequences may also be used including, but not limited to, simian virus 40 (SV40) early promoter, mouse mammary tumor virus (MMTV), human immunodeficiency virus (HIV) long terminal repeat (LTR) promoter, MoMuLV promoter, avian leukosis virus promoter, Epstein-Barr virus immediate early promoter, Rous sarcoma virus promoter, and human gene promoters such as, but not limited to, actin promoters, myosin promoters, heme promoters, and creatine kinase promoters. Further, the present invention should not be limited to the application of constitutive promoters. Inducible promoters are also contemplated as part of the present invention. The use of inducible promoters provides molecular switches that are capable of turning on expression of polynucleotide sequences operably linked to an inducible promoter when such expression is desired, or turning off expression when expression is undesired. Examples of inducible promoters include, but are not limited to, metallothionein promoters, glucocorticoid promoters, progesterone promoters, and tetracycline promoters.

In order to assess expression of the CAR polypeptide or portion thereof, the expression vector that is introduced into the cell may also comprise either or both of selectable marker genes or reporter genes to facilitate identification and selection of expressing cells from a population of cells seeking to be transfected or infected via a viral vector. In other aspects, the selectable marker may be carried on a separate segment of DNA and used in a cotransfection program. Both the selectable marker and the reporter gene may be flanked by appropriate regulatory sequences to enable expression in the host cell. Useful selectable markers include, for example, antibiotic resistance genes such as neo and the like.

The reporter gene is used to identify potentially transfected cells and for evaluating the functionality of the regulatory sequence. Typically, a reporter gene is a gene which is not present in or expressed by the recipient organism or tissue and which encodes a polypeptide, the expression of which is clearly indicated by some readily detectable property such as enzymatic activity. The expression of the reporter gene is measured at a suitable time after the DNA has been introduced into the recipient cell. Suitable reporter genes may include genes encoding luciferase, β-galactosidase, chloramphenicol acetyltransferase, secreted alkaline phosphatase, or green fluorescent protein (e.g., Ui-Tei et al., 2000 FEBS Letters 479:79-82). Suitable expression systems are well known and can be prepared using known techniques or are commercially available. Typically, constructs with a minimum of five flanking regions that show the highest level of reporter gene expression are identified as promoters. Such promoter regions can be ligated to the reporter gene and used to evaluate the ability of the reagent to regulate promoter-driven transcription.

Methods of introducing genes into cells and expressing genes into cells are known in the art. In the content of expression vectors, the vectors may be readily introduced into a host cell, e.g., a mammalian, bacterial, yeast, or insect cell, by any method in the art. For example, the expression vector may be transferred into the host cell by physical, chemical or biological means.

Physical methods for introducing a polynucleotide into a host cell include calcium phosphate precipitation, lipid transfection methods, particle bombardment, microinjection, electroporation, and the like. Methods for producing cells comprising vectors and/or exogenous nucleic acids are well known in the art. See, for example, Sambrook et al. (2001, Molecular Cloning: a Laboratory Manual, Cold Spring Harbor Laboratory, New York). A preferred method of introducing a polynucleotide into a host cell is calcium phosphate transfection.

Biological methods for introducing a polynucleotide of interest into a host cell include the use of DNA and RNA vectors. Viral vectors, particularly retroviral vectors, have become the most widely used method of inserting genes into mammalian, e.g. human, cells. Other viral vectors can be derived from lentiviruses, poxviruses, herpes simplex virus I, adenoviruses and adeno-associated viruses, among others. See, e.g., U.S. Pat. Nos. 5,350,674 and 5,585,362.

Chemical means of introducing polynucleotides into host cells include colloidal dispersion systems, such as macromolecular complexes, nanocapsules, microspheres, beads; and lipid-based systems, including oil-in-water emulsions, micelles, hybrid micelles, and liposomes. Exemplary colloidal systems used as in vitro and in vivo delivery vehicles are liposomes (e.g., artificial membrane vesicles).

In the case of using non-viral delivery systems, exemplary delivery vehicles are liposomes. The use of a lipid formulation is contemplated for introducing a nucleic acid into a host cell (in vitro, ex vivo (isolated), or in vivo). In another aspect, the nucleic acid may be associated with a lipid. The nucleic acid associated with a lipid may be encapsulated into the aqueous interior of a liposome, dispersed within the lipid bilayer of a liposome, attached to the liposome by a linker molecule associated with both the liposome and the oligonucleotide, trapped in the liposome, complexed with the liposome, dispersed in a solution comprising the lipid, mixed with the lipid, associated with the lipid, contained in the lipid as a suspension, contained in or complexed with micelles, or otherwise associated with the lipid. The lipids, lipids/DNA, or lipids/expression vectors associated with the compositions are not limited to any specific structure in solution. For example, they may be present in bilayer structures, as micelles or have a “collapsed” structure. They may also simply be dispersed in solution and may form aggregates of uneven size or shape. Lipids are fatty substances, which may be naturally occurring or synthetic. For example, lipids include fat droplets, which occur naturally in the cytoplasm as well as in compounds of the class comprising long chain aliphatic hydrocarbons and their derivatives such as fatty acids, alcohols, amines, amino alcohols and aldehydes.

In a preferred embodiment of the invention, said vector is a lentiviral vector.

### Formulations

The present invention provides a formulation comprising a CAR-T cell of the present invention, and a pharmaceutically acceptable carrier, diluent or excipient. In one embodiment, said formulation is a liquid formulation. Preferably, said formulation is an injection. Preferably, the concentration of said CAR-T cells in said formulation is 1×10⁵-1×10⁸ cells/ml, preferably 1×10⁷-1×10⁸ cells/ml.

In one embodiment, said formulation may comprise a buffer such as neutral buffered saline, sulfate buffered saline, and the like; carbohydrates such as glucose, mannose, sucrose or dextran, mannitol; proteins; peptides or amino acids such as glycine; antioxidants; chelating agents such as EDTA or glutathione; adjuvants (e.g., aluminum hydroxide); and preservatives. The formulations of the present invention are preferably formulated for intra-venous administration.

### Therapeutic Applications

The present invention includes therapeutic applications with cells (e.g., T cells) transduced with lentiviral vectors (LVs) encoding expression cassettes of the present invention. The transduced T cells target HER2 and MESO, markers of tumor cells, synergistically activating the T cells and eliciting a T cell immune response, thereby significantly increasing their killing efficiency against tumor cells.

Accordingly, the present invention also provides methods of stimulating a T cell-mediated immune response against a target cell population or tissue of a mammal comprising the steps of: administering to the mammal a CAR-T cell of the present invention.

In one embodiment, the present invention comprises a class of cell therapies in which patient autologous T cells (or heterologous donors) are isolated, activated and genetically modified to produce CAR-T cells, which are subsequently injected into the same patient. This approach has a very low probability of developing graft-versus-host disease, and the antigen is recognized by the T cells in an MHC-restriction-free manner. In addition, one CAR-T can treat all cancers that express the antigen. Unlike antibody therapies, CAR-T cells are capable of replicating in vivo, producing long term durability that can lead to sustained tumor control.

In one embodiment, the CAR-T cells of the present invention can undergo solid in vivo T cell expansion and can be sustained for extended amounts of time. Alternatively, the CAR-mediated immune response may be part of a step of adoptive immunotherapy in which the CAR-modified T cells induce an immune response specific to an antigen-binding domain in the CAR. For example, anti-HER2 and MESO CAR-T cells elicit a specific immune response against HER2 and/or MESO positive cells.

Although the data disclosed herein specifically discloses lentiviral vectors comprising a HER2-MESO scFv, a 4-1BB intracellular region, and a CD3ζ signaling domain, the present invention should be construed to encompass any number of variations to each of the construct components.

Hematologic cancers are cancers of the blood or bone marrow. Examples of hematological (or hematogenous) cancers include leukemias, including acute leukemias (such as acute lymphoblastic leukemia, acute myeloid leukemia, acute myelogenous leukemia, and myeloblastic, promyelocytic, granulocytic-monocytic, mononuclear cellular, and erythroleukemic leukemias), chronic leukemias (such as chronic myelomonocytic (granulocytic) leukemia, chronic myelogenous leukemia, and chronic lymphocytic leukemia), polycythemia vera, lymphoma, Hodgkin disease, non-Hodgkin lymphoma (painless and high-grade forms), multiple myeloma, Waldenstrăm's macroglobulinemia, heavy-chain disorders, myelodysplastic syndromes, hairy cell leukemia, and myelodysplasia.

Solid tumors are abnormal masses of tissue that do not usually contain cysts or areas of fluid. Solid tumors can be benign or malignant. The different types of solid tumors are named after the type of cells that form them (such as sarcomas, carcinomas, and lymphomas). Examples of solid tumors such as sarcomas and carcinomas include fibrosarcomas, myxomatodes sarcoma, liposarcoma mesothelioma, lymphoid malignancies, and pancreatic cancers, ovarian cancers.

In preferred embodiments, the treatable cancers are HER2 and/or MESO positive tumors such as pancreatic cancer.

The CAR-modified T cells of the present invention can also be used as a vaccine type for ex vivo immunization and/or in vivo therapy in mammals. Preferably, the mammal is human.

For ex vivo immunization, at least one of the following occurs in vitro prior to administration of the cells into the mammal: i) expansion of the cells, ii) introduction of nucleic acid encoding the CAR into the cells, and/or iii) cryopreservation of the cells.

The ex vivo procedure is well known in the art and is discussed more fully below. Briefly, cells are isolated from a mammal (preferably a human) and genetically modified (i.e., transduced or transfected in vitro) with a vector expressing a CAR disclosed herein. The CAR-modified cells may be administered to a mammalian recipient to provide a therapeutic benefit. The mammalian recipient may be human, and the CAR-modified cells may be autologous relative to the recipient. Optionally, the cells may be allogeneic, isoplastic (syngeneic), or heterogenous relative to the recipient.

In addition to the use of cell-based vaccines with respect to ex vivo immunization, the present invention also provides compositions and methods for in vivo immunization to elicit an immune response against an antigen in a patient.

The present invention provides methods of treating a tumor comprising administering to a subject in need thereof a therapeutically effective amount of CAR-modified T cells of the present invention.

The CAR-modified T cells of the present invention may be administered alone or as a pharmaceutical composition in combination with a diluent and/or with other components such as IL-2, IL-17, or other cytokines or cell populations. Briefly, the pharmaceutical compositions of the present invention may include target cell populations as described herein, in combination with one or more pharmaceutically or physiologically acceptable carriers, diluents or excipients. Such compositions may include buffers such as neutral buffered saline, sulfate buffered saline, and the like; carbohydrates such as glucose, mannose, sucrose or dextran, mannitol; proteins; peptides or amino acids such as glycine; antioxidants; chelating agents such as EDTA or glutathione; adjuvants (e.g., aluminum hydroxide); and preservatives. The compositions of the present invention are preferably formulated for intravesical administration.

The pharmaceutical compositions of the present invention may be administered in a manner suitable for the disease to be treated (or prevented). The amount and frequency of administration will be determined by such factors as the patient's condition, and the type and severity of the patient's disease—although the appropriate dosage may be determined by clinical trials.

When “immunologically effective amount”, “antitumor effective amount”, “tumor-inhibitory effective amount” or “therapeutic amount” is indicated, the precise amount of the composition of the present invention to be administered may be determined by a physician taking into account individual differences in age, weight, tumor size, degree of infection or metastasis, and condition of the patient (subject). It may be generally noted that: pharmaceutical compositions comprising the T cells described herein may be administered at a dose of 10⁴ to 10⁹ cells/kg of body weight, preferably 10⁵ to 10⁶ cells/kg of body weight (including all integer values within those ranges). the T cell compositions may also be administered multiple times at these doses. The cells may be administered by using injection techniques well known in immunotherapy (see, e.g., Rosenberg et al, New Eng. J. of Med. 319:1676, 1988). The optimal dosage and treatment regimen for a particular patient can be readily determined by a person skilled in the art of medicine by monitoring the patient for signs of disease and consequently modifying the treatment.

Administration of the subject compositions may be carried out in any convenient manner, including by spray method, injection, swallowing, infusion, implantation or transplantation. The compositions described herein may be administered to a patient subcutaneously, intradermally, intratumorally, intranodally, intraspinal, intramuscularly, by intravenously (i.v.) injection, or intraperitoneally. In one embodiment, the T cell compositions of the present invention are administered to a patient by intradermal or subcutaneous injection. In another embodiment, the T cell compositions of the present invention are preferably administered by i.v. injection. The compositions of T cells may be injected directly into a tumor, lymph node, or site of infection.

In certain embodiments of the present invention, cells activated and expanded using the methods described herein or other methods known in the art for expanding T cells to therapeutic levels are administered to a patient in combination with (e.g., prior to, concurrently with, or subsequent to) any number of related therapeutic forms, said therapeutic forms comprising, but not limited to, treatment with the following reagents: said reagents such as antiviral therapies, cidofovir and leukocyte interleukin-2, cytarabine (also known as ARA-C) or natalizumab therapy for MS patients or erfalizumab therapy for psoriasis patients or other therapies for PML patients. In further embodiments, the T cells of the present invention may be used in combination with: chemotherapy, radiation, immunosuppressive agents such as, for example, cyclosporine, azathioprine, methotrexate, meclofenoxate and FK506, antibodies or other immunotherapeutic agents. In further embodiments, the cellular compositions of the present invention are administered to a patient in combination (e.g., before, at the same time, or after) with a bone marrow transplant, utilizing a chemotherapeutic agent such as fludarabine, external beam radiation therapy (XRT), cyclophosphamide. For example, in one embodiment, the subject may undergo standard treatment with high-dose chemotherapy followed by peripheral blood stem cell transplantation. In some embodiments, following the transplant, the subject receives an infusion of the expanded immune cells of the present invention. In an additional embodiment, the expanded cells are administered before or after the surgical procedure.

The dosage of the above treatments administered to a patient will vary with the precise properties of the condition being treated and the recipient of the treatment. The ratio of doses administered to a person may be implemented according to practices accepted in the art. Typically, 1×10⁶ to 1×10¹⁰ modified T cells (CAR-T cells) of the present invention may be administered to a patient per treatment or per course of therapy, by, for example, intravenous infusion.

The main advantages of the present invention include:

(1) The bispecific CAR-T cells of the present invention have significant killing effects on HER2-positive target cells and MESO-positive target cells.

(2) The bispecific CAR-T cells of the present invention enhance the tumor-killing effect of T cells and cytokines produced have a super-additive effect, which can better clear tumor cells and mitigate the antigen escape phenomenon caused by tumor heterogeneity compared with single-target CAR-T, further enhancing the tumor-killing ability of CAR-T cells.

(3) The present invention finds for the first time that since the T cells can simultaneously tandemly express single-chain antibodies against the tumor-associated antigens HER2 and MESO on the surface of the pancreatic cancer cells, the range of tumor cells recognized by the T cells is greatly increased, and cancer cells below the recognition threshold of the single-targeted CAR-T cells are recognized, which facilitates the wide application of the tandem CAR-T cells in a heterogeneous subpopulation of tumors, this results in a wider range of killing of pancreatic cancer cells, at the same time, tandem CAR-T cells can enhance the tumor killing effect of T cells and cytokines produced have a super-additive effect, which can better clear tumor cells and alleviate the antigen escape phenomenon caused by tumor heterogeneity compared with single-target CAR-T, further strengthening the tumor killing ability of CAR-T cells.

The present invention is further described below in connection with specific embodiments. It should be understood that these embodiments are used only to illustrate the invention and are not intended to limit the scope of the invention. Experimental methods for which specific conditions are not indicated in the following embodiments are generally in accordance with conventional conditions, such as those described in Sambrook et al, Molecular Cloning: a Laboratory Manual (New York: Cold Spring Harbor Laboratory Press, 1989), or in accordance with conditions recommended by the manufacturer. Unless otherwise indicated, percentages and portions are weight percentages and weight portions.

The reagents and materials used in the Examples are commercially available products, if not otherwise indicated.

The present invention discloses HM CAR plasmids capable of tandemly expressing and targeting HER2 and MESO simultaneously, HM CAR-T cells, methods of constructing the same, and applications thereof. FIG. 2 shows a flowchart of the present invention, as shown in the following embodiments.

### Example 1

### Construction of HM CAR Plasmid Capable of Expressing Both HER2 Single Chain Antibody and MESO Single Chain Antibody

Sequentially ligated Signal Peptide-HER2V_{L}-(G4S)₃-HER2V_{H}-(G4S)₅-MESOV_{H}-(G4S)₃-MESOV_{L}-CD8α-4-1BB-CD3ζ. Sequences were ligated with BamHI and XhoI cleavage sites at both ends, respectively. All sequences were humanized, synthesized by Sangon Biotech (Shanghai) Co., Ltd. and preserved as PUC57 plasmid.

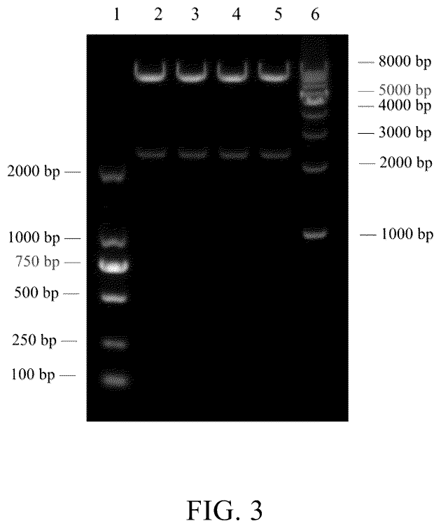

The obtained gene sequence fragments were ligated into the lentiviral expression vector pLenti6.3/V5 using an enzymatic ligation method to obtain the HM CAR plasmid. FIG. 3 shows the results of agarose gel electrophoresis of the HM CAR gene constructed in the pLenti6.3/V5 vector after identification by BamHI and XhoI double digestion.

### Example 2

### Lentiviral Packaging

After adjusting HEK293 cells to 2×10⁶ live cells/mL, 25.5 mL was taken and 1.5 mL of LV-MAX Supplement was added;

Prepare DNA/LV-MAX transfection reagent complex:

Test tube 1: labeled “DNA”

(1) Add 1.5 mL Opti-MEM serum-free medium;

(2) Add three helper plasmid mixtures (1.5 μg/mL) and lentiviral expression vector pLenti6.3/V5 (1 μg/mL);

Test tube 2: labeled “TfxR”

(1) Add 1.5 mL Opti-MEM serum-free medium;

(2) Add 180 μL of LV-MAX transfection reagent, vortex briefly and incubate at room temperature for 1 minute;

(3) After 1 minute, pour tube 1 to tube 2 or combine the two solutions in the opposite way and vortex briefly;

(4) After incubating the mixed solution for 10 minutes at room temperature, add the DNA/LV-MAX transfection complex directly to HEK293 cells;

(5) 5-6 hours after transfection, 1.2 mL of LV-MAX reinforcing agent was added to the cells.

After 48-55 hours of transfection, the lentiviral stock solution (HM CAR-LV) was pipetted to a 50 mL centrifuge tube and centrifuged at 1300 g for 15 min to collect the supernatant. The supernatant was then filtered through a 0.45 μm low protein binding filter to remove cellular debris, and concentrated by ultrafiltration at 4° C. in a 100 KD ultrafiltration tube to concentrate 30 mL of the system to 5 mL, aliquotting the lentiviral concentrate and storing it in a −80° C. refrigerator.

Real-time fluorescence quantitative PCR (qPCR) to detect lentivirus titer

(1) Treat 24-well plates with polylysine to prevent 293T cells from detaching during transfection;

(2) Spread 1 ml of plates with a density of 2×10⁵/ml cells and incubate overnight at 37° C. in an incubator containing 5% CO2;

(3) Dilute the lentivirus with serum-free medium, and dilute the lentivirus stock solution 10-fold, 100-fold, and 1,000-fold, respectively. The old medium was aspirated and 500 μL of lentiviral dilution and 10 μL of transfection agent were added to the cells to increase the transfection efficiency, and the culture was continued. On the next day, the cell culture medium was changed to DMEM medium containing 10% FBS;

(4) After 72 h of transfection, 293T cells were digested and cellular DNA was extracted, and CAR gene copy number was detected by qPCR. The titer of HM CAR-LV stock solution obtained is higher than 10⁷ TU/mL, and the titer can be as high as 10⁹ TU/mL after lentivirus concentration with 100 KD ultrafiltration tubes. The results are shown in the table below:

| | titer | | | |
|---|---|---|---|---|
| | | | | Mean titer |
| groups | 10⁻¹ | 10⁻² | 10⁻³ | (TU/mL) |
| HM | 1.91E+06 | 3.04E+05 | 2.31E+04 | 2.42E+07 |
| CAR-LV | | | | |
| NEG | — | — | — | 494.9 |

### Example 3

### CAR-T Cell Preparation

### PBMC Cell Recovery and Magnetic Bead Sorting

(1) Take human PBMC out of the liquid nitrogen tank carefully, put it into a 37° C. water bath, and transfer it into a safety cabinet after it has dissolved to a small ice cube;

(2) Pipette 5 mL of HBSS (containing 10% human albumin), then aspirate the cells into a 50 mL centrifuge tube, and lubricate the freezing tube with 5 mL of HBSS (containing 10% human albumin);

(3) Slowly add 30 mL of HBSS (containing 10% human albumin) to a total volume of 40 mL, centrifuged at 400 g for 10 min, and discard the supernatant;

(4) Add 1 mL of 1640 medium (containing 10% FBS) and 8 μL of DNAse, and left for 15 min at 37° C.;

(5) Add 29 mL of 1640 medium (containing 10% FBS) and left at 37°° C. for 4˜6 hours;

(6) After several hours of standing, perform cell counting to determine the total cell volume;

(7) Centrifuged at 300 g for 10 min, discard the supernatant, add a certain volume of MACs Buffer Running Buffer (Buffer) to resuspend the cells, and then add a certain volume of CD3 sorting beads, 4° C., centrifugation for 15 min; (Note: Suspend every 10⁷ cells in 80 μL of Buffer; add 20 μL of CD3 magnetic beads to every 10⁷ cells).

(8) Add a certain volume of Buffer, centrifuged at 300 g for 10 min, discard supernatant; (Note: add 2 mL Buffer per 10⁷ cells)

(9) Resuspend the cells again with a certain volume of Buffer; (Note: add 500 μL Buffer per 10⁷ cells)

(10) Perform column equilibration when centrifuging cells in step 8 (activate LS column with 3 mL Buffer);

(11) Add the resuspended cell suspension from step 9 to the column, gravity flow out and flow through and collect;

(12) Wash the column with Buffer, 3 mL at a time, three times and collect the flow-through;

(13) Remove the column from the separator and placed on a suitable collection tube;

(14) Elute the LS column with 5 mL Buffer and perform cell counting;

(15) At 300 g, centrifuged for 10 min, discard the supernatant, add TexMACs medium (containing IL-2) and adjust the cell concentration to 1×10⁶/mL;

(16) Add T cell CD3CD28 stimulant; (Note: add 10 μL of stimulant for every 10⁶ cells)

(17) Spread 24-well plates with 500 μL and incubated overnight at 37° C. to prepare for transfection of T cells on the next day.

### CAR-T Cell Preparation

(1) Lentivirus was used to transfect T cells with MOI 15, blowing and mixing;

(2) Then add Polybrene to enhance the transfection efficiency;

(3) Add 500 μL TexMACs medium after 4 h. (Note: Liquid change was performed every two days)

### Detection for the Positive Rate of CAR-T Cells

Take 1×10⁶ post-transduction T cells, incubated with biotin-labeled HER2 and Meso for 1 h at 4° C., avoid light, wash with PBS (containing 2% FBS) twice; add 100 μL PBS (containing 2% FBS) to resuspend the cells, add 10 μL PE-labeled avidin, incubated at 4° C., avoid light for 1 h, wash with PBS (containing 2% FBS) twice, and resuspend the cells with 600 μL PBS for on-boarding. The PE fluorescence signal was detected by flow cytometry, reflecting the positive rate of CAR-T cells in the total cells.

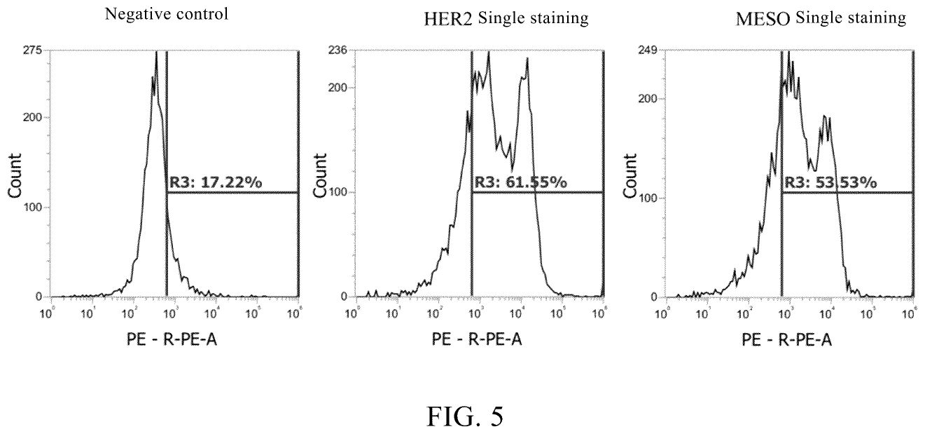

The detection results are shown in FIG. 5, from the results showing that the positive rate of HM CAR-T cells is about 50%.

The CAR gene in CAR-T cells was detected by real-time fluorescence quantitative PCR, which further proved the successful transfection of T cells by lentivirus at the gene level. 1×10⁶ HM CAR-T cells and untransfected T cells were taken respectively, cellular DNA was extracted, and CAR gene copy number was detected by qPCR.

The specific experimental results are shown in Table 1, which proves that the CAR gene is successfully integrated into the T cell genome.

TABLE 1
Real-time fluorescence quantitative PCR for
detection of cellular CAR gene

| | CAR | |
|---|---|---|
| | gene | copy |
| Groups | number | SD |
| HM CAR-T | 14.091 | 0.263 |
| NEG-T cell | 0.099 | 0.018 |

### Example 4

### In Vitro Killing of Pancreatic Cancer Cells by Dual-Target CAR-T Cells

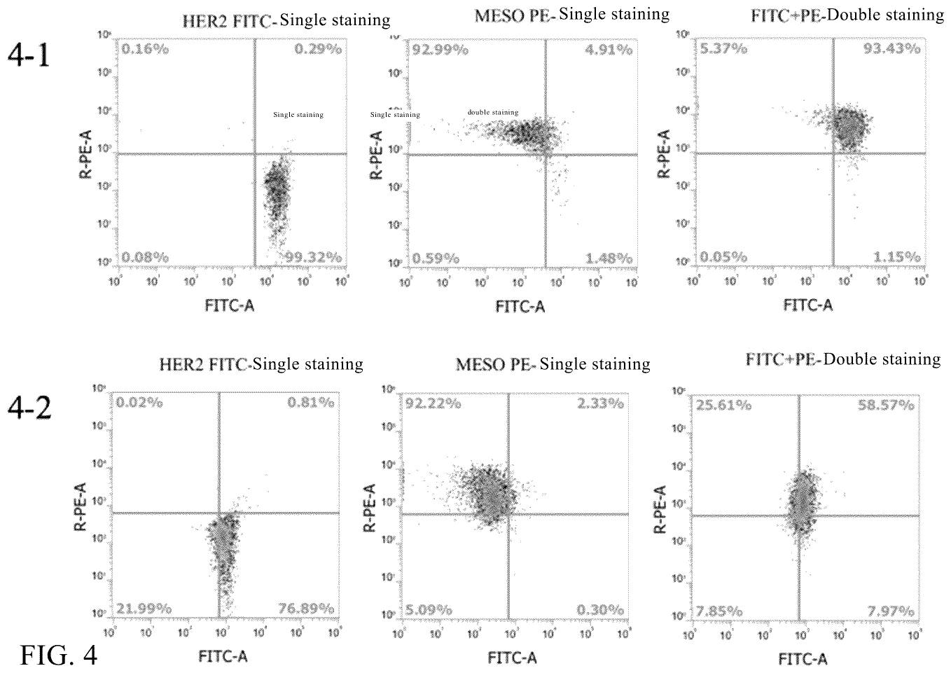

Pancreatic cancer cells SW-1990 and ASPC-1 surface HER2 and MESO antigen expression was detected by flow cytometric analysis, and its antigen-positive rate results are shown in FIG. 4.

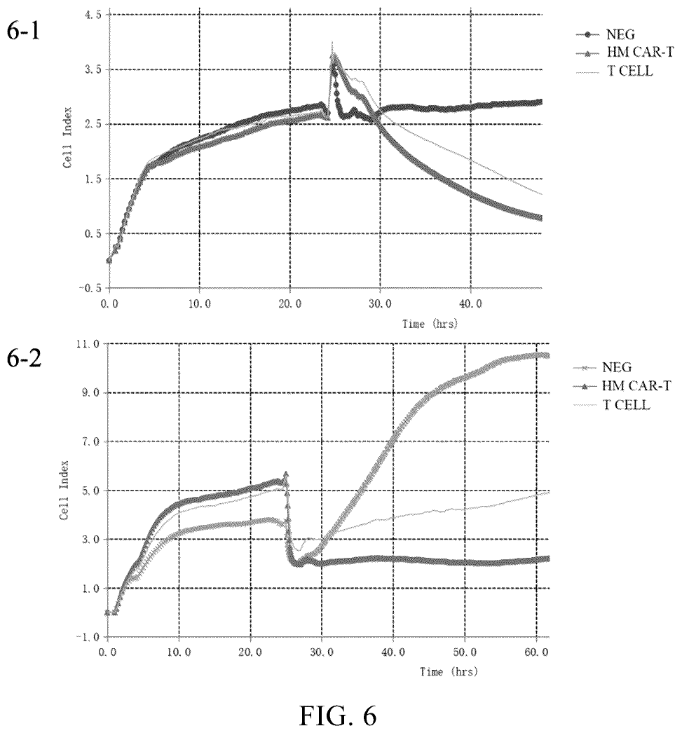

RTCA was used to detect HM CAR-T cell killing of the two pancreatic cancer cell lines, and the effector-target ratio of 4:1 was set for killing. Pancreatic cancer cells with 8×10⁴ ASPC-1 cells and 1×10⁴ SW-1990 were plated and cultured for 24 h, and then CAR-T cells and T cells with a effector-target ratio of 4:1 were added to compare the difference in tumor cell killing between the two. The results are shown in FIG. 6: 6-1 shows the killing of ASPC-1 by HM CAR-T cells, and 6-2 shows the killing of SW-1990 by HM CAR-T cells. The experimental results prove that the killing effect of HM CAR-T cells is more advantageous than that of the control group, indicating that the tandem HM CAR-T cells expressing both targeting HER2 and MESO are specifically targeted to kill HER2 and MESO positive pancreatic cancer cells.

The results show that HM CAR-T cells significantly delay tumor growth with p<0.05 compared to the control group (FIG. 6).

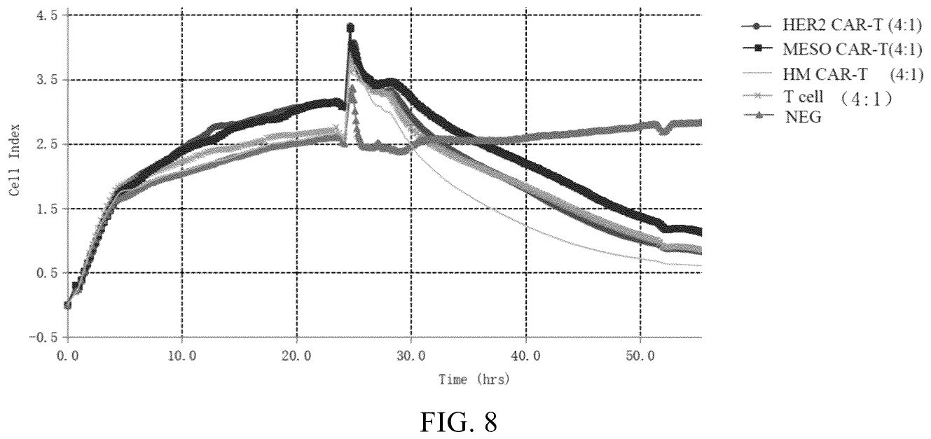

In addition, the killing effect of the three CAR-T cells on ASPC-1 cells was detected by RTCA. A total of 80K cells were plated, cultured for 24 h, and then CAR-T cells and T cells with a 4:1 effector-target ratio were added to compare the different killing effects of different cells on tumor cells. The experimental results show that compared with the negative control group, both T cells and CAR-T cells have different degrees of killing effect, but the killing effect of CAR-T cells in the tandem group is more advantageous. (FIG. 8)

All publications mentioned herein are incorporated by reference as if each individual document was cited as a reference in the present application. It should be understood that, after reading the above teachings of the present invention, those skilled in the art can make various modifications and changes. These equivalent forms are also within the scope defined by the claims appended hereto.

1. A bispecific chimeric antigen receptor (CAR), having a structure as shown in Formula I below:
L-scFv1-I-scFv2-H-TM-C-CD3ζ (I)
wherein
each “-” is independently a linker peptide or peptide bond;
L is an optional signal peptide sequence;
I is a flexible linker;
H is an optional hinge region;
TM is a transmembrane domain;
C is co-stimulatory signaling molecule;
CD3ζ is a cytoplasmic signaling sequence derived from CD3ζ;
One of the two, scFv1 and scFv2, is an antigen-binding domain targeting HER2 and the other is an antigen-binding domain targeting MESO.

2. The bispecific chimeric antigen receptor of claim 1, wherein the scFv1 is an antigen-binding domain targeting HER2 and the scFv2 is an antigen-binding domain targeting MESO.

3. The bispecific chimeric antigen receptor of claim 1, wherein the antigen-binding domain targeting HER2 has a structure as shown in Formula A or Formula B below:
V_{H1}-V_{L1} (A);
V_{L1}-V_{H1} (B)
wherein V_{H1} is a heavy chain variable region; of an anti-HER2 antibody; V_{L1} is a light chain variable region of an anti-HER2 antibody; and “-” is a linker peptide or peptide bond.

4. A nucleic acid molecule encoding the chimeric antigen receptor (CAR) of claim 1.

5. A vector comprising a nucleic acid molecule of claim 4.

6. A host cell containing a vector of claim 5.

7. A formulation comprising the chimeric antigen receptor of claim 1 and a pharmaceutically acceptable carrier, diluent or excipient.

8. Use of the chimeric antigen receptor of claim 1 for the preparation of a medicament or a formulation for the prevention and/or treatment of a cancer or a tumor.

9. A kit for preparing a host cell, comprising a container, and the nucleic acid molecule of claim 4 in the container.

10. A method of preparing an engineered immune cell expressing the CAR of claim 1, comprising:
(a) providing an immune cell to be engineered; and
(b) transducing a nucleic acid molecule encoding the CAR of claim 1 or a vector comprising the nucleic acid molecule encoding the CAR of claim 1 into the immune cell, thereby obtaining the engineered immune cell.

11. A host cell containing a chromosome incorporating an exogenous nucleic acid molecule of claim 4.

12. A host cell expressing a CAR of claim 1.

13. A formulation comprising the nucleic acid molecule of claim 4 and a pharmaceutically acceptable carrier, diluent or excipient.

14. A formulation comprising the vector of claim 5 and a pharmaceutically acceptable carrier, diluent or excipient.

15. A formulation comprising the host cell of claim 6 and a pharmaceutically acceptable carrier, diluent or excipient.

16. A kit for preparing a host cell, comprising a container and the nucleic acid molecule of claim 4 in the container.
